SEQUENCE LISTING (34 sequences)

SEQ ID NO: 1 - DNA, 26 bp, Artificial Sequence
  Synthetic Sequence
tggattatgcacttgaaatatgtgag

SEQ ID NO: 2 - DNA, 26 bp, Artificial Sequence
  Synthetic Sequence
tggattatgcacttgaaatatgtgaa

SEQ ID NO: 3 - DNA, 20 bp, Artificial Sequence
  Synthetic Sequence
tggtgttgccatttccactg

SEQ ID NO: 4 - DNA, 26 bp, Artificial Sequence
  Synthetic Sequence
atatcaagatatgtagctgctatagc

SEQ ID NO: 5 - DNA, 26 bp, Artificial Sequence
  Synthetic Sequence
atatcaagatatgtagctgctatagt

SEQ ID NO: 6 - DNA, 20 bp, Artificial Sequence
  Synthetic Sequence
tttcgactttcttctggtca

SEQ ID NO: 7 - DNA, 20 bp, Artificial Sequence
  Synthetic Sequence
aggtaatcatttacgaatcg

SEQ ID NO: 8 - DNA, 20 bp, Artificial Sequence
  Synthetic Sequence
aggtaatcatttacgaatca

SEQ ID NO: 9 - DNA, 25 bp, Artificial Sequence
  Synthetic Sequence
tctgacataagaaaatcctccttgt

SEQ ID NO: 10 - DNA, 24 bp, Artificial Sequence
  Synthetic Sequence
agccaggttcttcatttaagatgg

SEQ ID NO: 11 - DNA, 24 bp, Artificial Sequence
  Synthetic Sequence
agccaggttcttcatttaagatgc

SEQ ID NO: 12 - DNA, 22 bp, Artificial Sequence
  Synthetic Sequence
ggtttcctctaccatggttcca

SEQ ID NO: 13 - DNA, 23 bp, Artificial Sequence
  Synthetic Sequence
gtcaattttacaattgttgacaa

SEQ ID NO: 14 - DNA, 23 bp, Artificial Sequence
  Synthetic Sequence
gtcaattttacaattgttgacac

SEQ ID NO: 15 - DNA, 22 bp, Artificial Sequence
  Synthetic Sequence
aggatcaaattcctcaaactca

SEQ ID NO: 16 - DNA, 26 bp, Artificial Sequence
  Synthetic Sequence
tcttctcttttgggtatattagttgc

SEQ ID NO: 17 - DNA, 26 bp, Artificial Sequence
  Synthetic Sequence
tcttctcttttgggtatattagttga

SEQ ID NO: 18 - DNA, 20 bp, Artificial Sequence
  Synthetic Sequence
agcagctggattttctgggt

SEQ ID NO: 19 - DNA, 25 bp, Artificial Sequence
  Synthetic Sequence
tcgcataaaggtattgcaaaacttg

SEQ ID NO: 20 - DNA, 25 bp, Artificial Sequence
  Synthetic Sequence
tcgcataaaggtattgcaaaactta

SEQ ID NO: 21 - DNA, 21 bp, Artificial Sequence
  Synthetic Sequence
aggtcaaaaacaggaagtgga

SEQ ID NO: 22 - DNA, 21 bp, Artificial Sequence
  Synthetic Sequence
acctcctagtatattattgag

SEQ ID NO: 23 - DNA, 21 bp, Artificial Sequence
  Synthetic Sequence
acctcctagtatattattgaa

SEQ ID NO: 24 - DNA, 20 bp, Artificial Sequence
  Synthetic Sequence
aaaggtggaggttttaagga

SEQ ID NO: 25 - DNA, 21 bp, Artificial Sequence
  Synthetic Sequence
agagtctccaagcatttcagt

SEQ ID NO: 26 - DNA, 21 bp, Artificial Sequence
  Synthetic Sequence
agagtctccaagcatttcagc

SEQ ID NO: 27 - DNA, 21 bp, Artificial Sequence
  Synthetic Sequence
actactgctggtcatggttct

SEQ ID NO: 28 - DNA, 22 bp, Artificial Sequence
  Synthetic Sequence
tccatcagttcttgcaaagtac

SEQ ID NO: 29 - DNA, 22 bp, Artificial Sequence
  Synthetic Sequence
tccatcagttcttgcaaagtag

SEQ ID NO: 30 - DNA, 22 bp, Artificial Sequence
  Synthetic Sequence
tccatcagttcttgcaaagtat

SEQ ID NO: 31 - DNA, 20 bp, Artificial Sequence
  Synthetic Sequence
gcttggtgcaacagataagt

SEQ ID NO: 32 - DNA, 26 bp, Artificial Sequence
  Synthetic Sequence
actttttattgcttattacttcattg

SEQ ID NO: 33 - DNA, 26 bp, Artificial Sequence
  Synthetic Sequence
actttttattgcttattacttcattt

SEQ ID NO: 34 - DNA, 24 bp, Artificial Sequence
  Synthetic Sequence
aggtgagtgtgaatgttcatttga

(19) United States
(12) Patent Application Publication
(10) Pub. No.: US 2022/0119866 A1
(43) Pub. Date: Apr. 21, 2022

(54) SNP MARKERS OF DRUG REDUCED SUSCEPTIBILITY RELATED EVOLUTIONARY BRANCHES OF CLOSTRIDIUM DIFFICILE, METHOD FOR IDENTIFYING STRAIN CATEGORY, AND USE THEREOF

(71) Applicant: BGI SHENZHEN, Shenzhen (CN)

(72) Inventors: Junhua LI, Shenzhen, Guangdong (CN); Hailong ZHAO, Shenzhen, Guangdong (CN); Ye PENG, Shenzhen, Guangdong (CN); Peter M. SHAW, Shenzhen, Guangdong (CN); David NICKLE, Shenzhen, Guangdong (CN); Judong SHEN, Shenzhen, Guangdong (CN)

(21) Appl. No.: 17/558,626

(22) Filed: Dec. 22, 2021

Related U.S. Application Data
(63) Continuation of application No. PCT/CN2019/092585, filed on Jun. 24, 2019.

(51) Int. Cl. C12Q 1/689

(52) U.S. Cl. CPC C12Q 1/689; G01N 2333/33; C12Q 2600/156

(57) ABSTRACT

Provided are SNP markers of drug reduced susceptibility related evolutionary branches of Clostridium difficile, a method for identifying the category of a Clostridium difficile strain, and use thereof. The SNP markers are specific markers of three categories of the Clostridium difficile clade2 (mainly hypervirulent ribotype 027), allowing for rapid and accurate identification of the evolutionary branches of Clostridium difficile strains that are resistant to a variety of therapeutic drugs and related drugs. Accurate categorization of the drug reduced susceptibility related evolutionary branches not only provides evidence for the evolutionary traceability of drug-resistant pathogens, but also offers effective and actionable guidance on clinical drug usage.

### CROSS-REFERENCE TO RELATED APPLICATIONS

This application is a continuation of International Application No. PCT/CN2019/092585, filed on Jun. 24, 2019, the disclosures of which hereby incorporated by reference in its entirety.

### FIELD

The present disclosure relates to the technical field of microbial drug resistance, in particular to SNP markers of drug reduced susceptibility related evolutionary branches of Clostridium difficile, a method for identifying the category of a Clostridium difficile strain, and use thereof.

### BACKGROUND

According to data released by the Centers for Disease Control and Prevention (CDC), USA in 2015, nearly 500,000 people in the United States are infected with Clostridium difficile each year, and the annual mortality is as high as 30,000 deaths. The bacterium has become the most common hospital-acquired pathogen instead of MRSA, the cost of treatment amounting to $1.5 billion per year. The total number of Clostridium difficile infections in Asians has also increased year by year. The bacteria has high resistance to common antibiotics such as erythromycin, clindamycin and fluoroquinolone, and can only be treated by metronidazole and vancomycin, the sensitivity to which the bacteria shows, however, has been ever-decreasing in recent years, particularly in the case of Clostridium difficile clade2 strains. Clade2 is defined according to the Clostridium difficile multilocus sequence typing (MLST) database (http://pubmlst.org/edifficile). The database determines the sequence type (ST) of each strain based on the polymorphism of seven Clostridium difficile housekeeping genes, and assigns the ST to one of five clades (clade1 to clade5) based on the evolutionary relationship within the whole species. As one of the clades, clade2 mainly comprises hypervirulent ribotype 027 (Ribotype027). As one of the 600 ribotypes of Clostridium difficile, Ribotype027 was experimentally obtained by taxonomic categorization by means of a polymerase chain reaction method using the polymorphism of the intergenic regions of 16S-23S ribosomal RNA genes, and has received much attention due to its high degree of drug resistance and clinical severity.

The continued use of antibiotics against the drug-resistant bacteria not only fails to achieve therapeutic effects, but also wastes resources and delays the clinical cycle, and may even cause more serious drug-resistant mutations. Existing techniques for detecting the drug resistance of the pathogen mainly include the following two means: (1) identification of the drug resistance by drug sensitivity test; and (2) identification of the drug resistance using existing detection kits of drug resistant determinants. The existing techniques for detecting the drug resistance of the pathogen suffer from the following problems: (1) detection of Clostridium difficile by drug sensitivity test involves stringent culture conditions and prolonged experimental period, which fails to meet the requirements of clinically rapid detection; and (2) the existing drug resistance detection kits do not allow for detection of therapeutic drugs and are thus of little significance in terms of clinical guidance.

### SUMMARY

The present disclosure provides SNP markers of drug reduced susceptibility related evolutionary branches of Clostridium difficile, a method for identifying the category of a Clostridium difficile strain, and use thereof. The present disclosure allows for rapid and accurate identification of the evolutionary branches of Clostridium difficile strains that are resistant to a variety of therapeutic drugs and related drugs, providing meaningful clinical therapeutic guidance.

According to a first aspect, an embodiment provides SNP markers of drug reduced susceptibility related evolutionary branches of Clostridium difficile, wherein the Clostridium difficile is the Clostridium difficile clade 2, and the SNP markers are selected one or more from the group consisting of the SNP markers in the following three categories, and combinations thereof:

(a) Category 1: an A base at genomic base position 1029237, a T base at genomic base position 1205938, an A base at genomic base position 2487991, an A base at genomic base position 2861888, a T base at genomic base position 882348, a G base at genomic base position 1798870, and an A base at genomic base position 3083454;

(b) Category 2: a C base at genomic base position 6310, and an A base at genomic base position 1550363;

(c) Category 3: a G base at genomic base position 118669, a C base at genomic base position 1205250, a C base at genomic base position 1235096, an A base at genomic base position 1462869, a T base at genomic base position 1549858, an A base at genomic base position 2367860, an A base at genomic base position 2851331, a C base at genomic base position 3031309, a G base at genomic base position 3419928, a G base at genomic base position 1602810, and a T base at genomic base position 2585036.

In a preferred embodiment, the Clostridium difficile clade 2 is hypervirulent ribotype 027 (Ribotype027).

According to a second aspect, an embodiment provides a method for identifying the category of a Clostridium difficile strain, comprising obtaining base information at the site of at least one SNP marker of the SNP markers according to the first aspect in a Clostridium difficile strain to be identified; and determining the category of the Clostridium difficile strain according to the base information.

In a preferred embodiment, the method comprises obtaining base information at the site of at least one SNP marker in Category 1, Category 2 or Category 3 according to the first aspect in a Clostridium difficile strain to be identified; and determining whether the Clostridium difficile strain belongs to said Category 1, Category 2 or Category 3 according to the base information.

In a preferred embodiment, the method comprises obtaining base information at the site of at least one SNP marker in each category of at least two categories of Category 1, Category 2 or Category 3 according to the first aspect in a Clostridium difficile strain to be identified; and determining whether the Clostridium difficile strain belongs to said Category 1, Category 2 or Category 3 according to the base information.

In a preferred embodiment, the method obtains the base information of the site of said SNP marker by amplifying a genomic region in which the site of said SNP marker is located by using primers adapted to specifically amplify the region, followed by sequencing the region.

In a third aspect, an embodiment provides a method for diagnosing the category of a Clostridium difficile strain in a subject infected with the strain, comprising: obtaining base information at the site of at least one SNP marker of the SNP markers according to the first aspect in the Clostridium difficile strain from the subject; and determining the category of the Clostridium difficile strain according to the base information.

In a preferred embodiment, the method comprises obtaining base information at the site of at least one SNP marker in Category 1, Category 2 or Category 3 according to the first aspect in the Clostridium difficile strain from the subject; and determining whether the Clostridium difficile strain belongs to said Category 1, Category 2 or Category 3 according to the base information.

In a preferred embodiment, the method comprises obtaining base information at the site of at least one SNP marker in each category of at least two categories of Category 1, Category 2 and Category 3 according to the first aspect in the Clostridium difficile strain from the subject; and determining whether the Clostridium difficile strain belongs to said Category 1, Category 2 or Category 3 according to the base information.

In a preferred embodiment, the method obtains the base information of the site of said SNP marker by amplifying a genomic region in which the site of said SNP marker is located by using primers adapted to specifically amplify the region, followed by sequencing the region.

In a fourth aspect, an embodiment provides a method for treating a subject infected with a Clostridium difficile strain, comprising obtaining base information at the site of at least one SNP marker of the SNP markers according to the first aspect in the Clostridium difficile strain from the subject; determining the category of the Clostridium difficile strain according to the base information; and administering moxifloxacin and/or metronidazole to the subject when the category of Clostridium difficile strain comprises Category 2 described above; and optionally, administering vancomycin to the subject when the category of the Clostridium difficile strain comprises Category 2 and/or Category 3 described above.

In a preferred embodiment, the method obtains the base information of the site of said SNP marker by amplifying a genomic region in which the site of said SNP marker is located by using primers adapted to specifically amplify the region, followed by sequencing the region.

In a fifth aspect, an embodiment provides primers adapted to specifically amplify a genomic region in which the site of a SNP marker according to the first aspect is located, wherein the primers comprise a forward primer and a reverse primer, the forward primer and the reverse primer respectively specifically binding to a genomic sense strand and a genomic anti sense strand flanking said SNP marker.

In a preferred embodiment, the primers comprise a fluorescent label and are adapted for fluorescence quantitative PCR.

In a preferred embodiment, the primers serve as hybridization probes to be immobilized on a chip to capture a sequence of the genomic region in which the site of said SNP marker is located.

In a sixth aspect, an embodiment provides a use of the primers according to the fifth aspect in the identification of the category of a Clostridium difficile strain, or in the diagnosis of the category of a Clostridium difficile strain from a subject infected with the strain.

In a seventh aspect, an embodiment provides a use of the SNP markers according to the first aspect in the identification of the category of a Clostridium difficile strain, or in the diagnosis of the category of a Clostridium difficile strain from a subject infected with the strain.

In an eight aspect, an embodiment provides a kit comprising primers, the kit comprises the primers comprising a forward primer and a reverse primer, the forward primer and the reverse primer respectively specifically binding to a genomic sense strand and a genomic antisense strand flanking a SNP marker according to the first aspect, and adapted to specifically amplify a genomic region in which the site of said SNP marker is located; optionally, the kit further comprises PCR amplification components besides the primers described above, such as a Taq DNA polymerase, dNTPs, and a reaction buffer, among others.

The SNP markers of drug reduced susceptibility related evolutionary branches of Clostridium difficile provided in the present disclosure are not only drug resistance markers, but also markers for identifying the drug reduced susceptibility related evolutionary branches of the Clostridium difficile clade 2 (mainly hypervirulent ribotype 027 (Ribotype027)). Strains in the same evolutionary branch have a closer phylogenetic relationship genome-wide, that is, share more identical genomic characteristics. The present disclosure allows for rapid and accurate identification of the evolutionary branches of Clostridium difficile resistant to a variety of therapeutic drugs and related drugs, providing meaningful clinical therapeutic guidance.

### BRIEF DESCRIPTION OF DRAWINGS

FIG. 1 is a diagram showing the evolutionary relationship and categorization of 269 strains of Clostridium difficile hypervirulent ribotype 027 (RT027) according to an example of the present disclosure.

### DESCRIPTION OF EMBODIMENTS

The present disclosure will be further described in detail below with reference to the accompanying drawings. In the following embodiments, many details are described so that the present disclosure will be better understood. However, those skilled in the art can readily recognize that some of the features may be omitted, or replaced by other materials or methods, depending on different situations.

Additionally, the characteristics, operations or features described in the specification can be combined in any suitable manner to form various embodiments. Moreover, the steps or actions in the description of the method may also be switched or adjusted in sequence in a manner that is obvious to those skilled in the art. Therefore, the various sequences in the description and the drawings are merely for the purpose of clearly describing a particular embodiment and are not intended to be required, unless it is otherwise specified that a specific sequence must be followed.

The present disclosure provides SNP markers of drug reduced susceptibility related evolutionary branches of Clostridium difficile, wherein the Clostridium difficile is the Clostridium difficile clade 2 (mainly hypervirulent ribotype 027 (Ribotype027)), and the SNP markers are selected one or more from the group consisting of the SNP markers in the following three categories, and combinations thereof:

(a) Category 1: an A base at genomic base position 1029237, a T base at genomic base position 1205938, an A base at genomic base position 2487991, an A base at genomic base position 2861888, a T base at genomic base position 882348, a G base at genomic base position 1798870, and an A base at genomic base position 3083454;

(b) Category 2: a C base at genomic base position 6310, and an A base at genomic base position 1550363;

(c) Category 3: a G base at genomic base position 118669, a C base at genomic base position 1205250, a C base at genomic base position 1235096, an A base at genomic base position 1462869, a T base at genomic base position 1549858, an A base at genomic base position 2367860, an A base at genomic base position 2851331, a C base at genomic base position 3031309, a G base at genomic base position 3419928, a G base at genomic base position 1602810, and a T base at genomic base position 2585036.

It should be noted that all of the above base position numbers are based on Clostridium difficile CD196 (NC_013315.1) as a reference genome.

Of the above-said 20 SNP markers in the present disclosure, 15 SNP markers have 100% category specificity. That is, the first 4 SNP markers in Category 1 only exist in the Clostridium difficile high-toxic ribotype 027 belonging to Category 1, both of the SNP markers in Category 2 only exist in the Clostridium difficile high-toxic ribotype 027 belonging to Category 2, and the first 9 SNP markers in Category 3 only exist in the Clostridium difficile high-toxic ribotype 027 belonging to Category 3. Therefore, by identifying the above-said SNP markers, it can be determined to which specific category of Category 1, Category 2 or Category 3 the Clostridium difficile hypervirulent ribotype 027 to be identified belongs.

Of the above-said 20 SNP markers in the present disclosure, the remaining 5 SNP markers besides the 15 SNP markers immediately described above have greater than 90% category specificity. That is, (1) for Category 1, when a T base is present at genomic base position 882348, the strain has a 93.3% possibility of belonging to Category 1, and when a C base is present at genomic base position 882348, the strain has a 100% possibility of belonging to Category 2 or Category 3; when a G base is present at genomic base position 1798870, the strain has a 91.8% possibility of belonging to Category 1, and when an A base is present at genomic base position 1798870, the strain has a 100% possibility of belonging to Category 2 or Category 3; and when an A base is present at genomic base position 3083454, the strain has a 96.6% possibility of belonging to Category 1, and when a G base is present at genomic base position 3083454, the strain has a 100% possibility of belonging to Category 2 or Category 3; and (2) for Category 3, when a G or T base is present at genomic base position 1602810, the strain has a 100% possibility of belonging to Category 3, and when a C base is present at genomic base position 1602810, the strain has a 100% possibility of belonging to Category 1 or Category 2; and when a T base is present at genomic base position 2585036, the strain has a 100% possibility of belonging to Category 3, and when a G base is present at genomic base position 2585036, the strain has a 95.2% possibility of belonging to Category 1 or Category 2.

It should be noted that by “any combination of SNP markers” is meant a combination of the SNP markers selected from any 1, 2 or 3 categories of the above-said three categories. For example, provided is a combination of any 1, 2, 3, 4, 5, 6 or 7 SNP markers selected from Category 1, such as, from Category 1, a combination of the SNP markers at genomic base positions 1029237 and 1205938, a combination of the SNP markers at genomic base positions 1029237 and 2487991, a combination of the SNP markers at genomic base positions 1029237 and 2861888, a combination of the SNP markers at genomic base positions 1029237 and 882348, a combination of the SNP markers at genomic base positions 1029237 and 1798870, a combination of the SNP markers at genomic base positions 1029237 and 3083454, a combination of the SNP markers at genomic base positions 1205938 and 2487991, a combination of the SNP markers at genomic base positions 1205938 and 2861888, a combination of the SNP markers at genomic base positions 1205938 and 882348, a combination of the SNP markers at genomic base positions 1205938 and 1798870, a combination of the SNP markers at genomic base positions 1205938 and 3083454, a combination of the SNP markers at genomic base positions 2487991 and 2861888, a combination of the SNP markers at genomic base positions 2487991 and 882348, a combination of the SNP markers at genomic base positions 2487991 and 1798870, a combination of the SNP markers at genomic base positions 2487991 and 3083454, a combination of the SNP markers at genomic base positions 2861888 and 882348, a combination of the SNP markers at genomic base positions 2861888 and 1798870, a combination of the SNP markers at genomic base positions 2861888 and 3083454, a combination of the SNP markers at genomic base positions 882348 and 1798870, a combination of the SNP markers at genomic base positions 882348 and 3083454, a combination of the SNP markers at genomic base positions 1798870 and 3083454; or a combination having 3, 4, 5, 6 or 7 SNP markers, formed on the basis of the above-said combinations by optionally incorporating a 3^{rd}, 4^{th}, 5^{th}, 6^{th} or 7^{th} SNP marker.

And for example, from Category 2, provided is a combination of the SNP markers at genomic base positions 6310 and 1550363.

Further for example, provided is a combination of any 1, 2, 3, 4, 5, 6, 7, 8, 9, 10, or 11 SNP markers from Category 3, such as a combination of the SNP markers at genomic base positions 118669 and 1205250, a combination of the SNP markers at genomic base positions 118669 and 1235096, a combination of the SNP markers at genomic base positions 118669 and 1462869, a combination of the SNP markers at genomic base positions 118669 and 1549858, a combination of the SNP markers at genomic base positions 118669 and 2367860, a combination of the SNP markers at genomic base positions 118669 and 2851331, a combination of the SNP markers at genomic base positions 118669 and 3031309, a combination of the SNP markers at genomic base positions 118669 and 3419928, a combination of the SNP markers at genomic base positions 118669 and 1602810, a combination of the SNP markers at genomic base positions 118669 and 2585036, a combination of the SNP markers at genomic base positions 1205250 and 1235096, a combination of the SNP markers at genomic base positions 1205250 and 1462869, a combination of the SNP markers at genomic base positions 1205250 and 1549858, a combination of the SNP markers at genomic base positions 1205250 and 2367860, a combination of the SNP markers at genomic base positions 1205250 and 2851331, a combination of the SNP markers at genomic base positions 1205250 and 3031309, a combination of the SNP markers at genomic base positions 1205250 and 3419928, a combination of the SNP markers at genomic base positions 1205250 and 1602810, a combination of the SNP markers at genomic base positions 1205250 and 2585036, a combination of the SNP markers at genomic base positions 1235096 and 1462869, a combination of the SNP markers at genomic base positions 1235096 and 1549858, a combination of the SNP markers at genomic base positions 1235096 and 2367860, a combination of the SNP markers at genomic base positions 1235096 and 2851331, a combination of the SNP markers at genomic base positions 1235096 and 3031309, a combination of the SNP markers at genomic base positions 1235096 and 3419928, a combination of the SNP markers at genomic base positions 1235096 and 1602810, a combination of the SNP markers at genomic base positions 1235096 and 2585036, a combination of the SNP markers at genomic base positions 1462869 and 1549858, a combination of the SNP markers at genomic base positions 1462869 and 2367860, a combination of the SNP markers at genomic base positions 1462869 and 2851331, a combination of the SNP markers at genomic base positions 1462869 and 3031309, a combination of the SNP markers at genomic base positions 1462869 and 3419928, a combination of the SNP markers at genomic base positions 1462869 and 1602810, a combination of the SNP markers at genomic base positions 1462869 and 2585036, a combination of the SNP markers at genomic base positions 1549858 and 2367860, a combination of the SNP markers at genomic base positions 1549858 and 2851331, a combination of the SNP markers at genomic base positions 1549858 and 3031309, a combination of the SNP markers at genomic base positions 1549858 and 3419928, a combination of the SNP markers at genomic base positions 1549858 and 1602810, a combination of the SNP markers at genomic base positions 1549858 and 2585036, a combination of the SNP markers at genomic base positions 2367860 and 2851331, a combination of the SNP markers at genomic base positions 2367860 and 3031309, a combination of the SNP markers at genomic base positions 2367860 and 3419928, a combination of the SNP markers at genomic base positions 2367860 and 1602810, a combination of the SNP markers at genomic base positions 2367860 and 2585036, a combination of the SNP markers at genomic base positions 2851331 and 3031309, a combination of the SNP markers at genomic base positions 2851331 and 3419928, a combination of the SNP markers at genomic base positions 2851331 and 1602810, a combination of the SNP markers at genomic base positions 2851331 and 2585036, a combination of the SNP markers at genomic base positions 3031309 and 3419928, a combination of the SNP markers at genomic base positions 3031309 and 1602810, a combination of the SNP markers at genomic base positions 3031309 and 2585036, a combination of the SNP markers at genomic base positions 3419928 and 1602810, a combination of the SNP markers at genomic base positions 3419928 and 2585036, a combination of the SNP markers at genomic base positions 1602810 and 2585036; or a combination having 3, 4, 5, 6, 7, 8, 9, 10, or 11 SNP markers, formed on the basis of the above-said combinations by optionally incorporating a 3^{rd}, 4^{th}, 5^{th}, 6^{th}, 7^{th}, 8^{th}, 9^{th}, 10^{th}, or 11^{th} SNP marker.

Typical but not limited examples of cross-category combinations of SNP markers include: a combination of any of 1, 2, 3, 4, 5, 6, or 7 SNP markers from Category 1 with any of 1 or 2 SNP markers from Category 2; or a combination of any of 1, 2, 3, 4, 5, 6, or 7 SNP markers from Category 1 with any of 1, 2, 3, 4, 5, 6, 7, 8, 9, 10, or 11 SNP markers from Category 3; or a combination of any of 1 or 2 SNP markers from Category 2 with any of 1, 2, 3, 4, 5, 6, 7, 8, 9, 10, or 11 SNP markers from Category 3; or a combination of any of 1, 2, 3, 4, 5, 6, or 7 SNP markers from Category 1 with any of 1 or 2 SNP markers from Category 2 and with any of 1, 2, 3, 4, 5, 6, 7, 8, 9, 10, or 11 SNP markers from Category 3.

In the present disclosure, based on the specimens of Clostridium difficile hypervirulent ribotype 027 collected from 16 countries around the world (a total of 269 strains), the evolutionary relationship of the strains of the Clostridium difficile hypervirulent ribotype 027 is constructed by means of whole genome sequencing, SNP calling, among other approaches, and evolutionary branches with significantly different proportions of drug-resistance and their corresponding markers are identified. RT027 is the principal ribotype in clade2. Cross distribution of RT027 along with other ribotypes such as RT198 and RT176 occurs in the whole-genome phylogenetic tree. Further, using the markers obtained, cluster analysis of clade2 and statistics of proportions of drug-resistance are conducted, revealing high consistency with RT027 grouping, because the markers can distinguish the reduced susceptibility related evolutionary branches in the whole clade2. Specifically, the work involves the following procedures:

(1) All of the strains are subjected to drug sensitivity test using three types of drugs (moxifloxacin MOX, metronidazole MET, vancomycin VAN, of which moxifloxacin belongs to fluoroquinolone antibiotics, closely related to the outbreak of Clostridium difficile, the latter two are the currently available therapeutic drugs against Clostridium difficile) to obtain resistant or sensitive phenotypes.

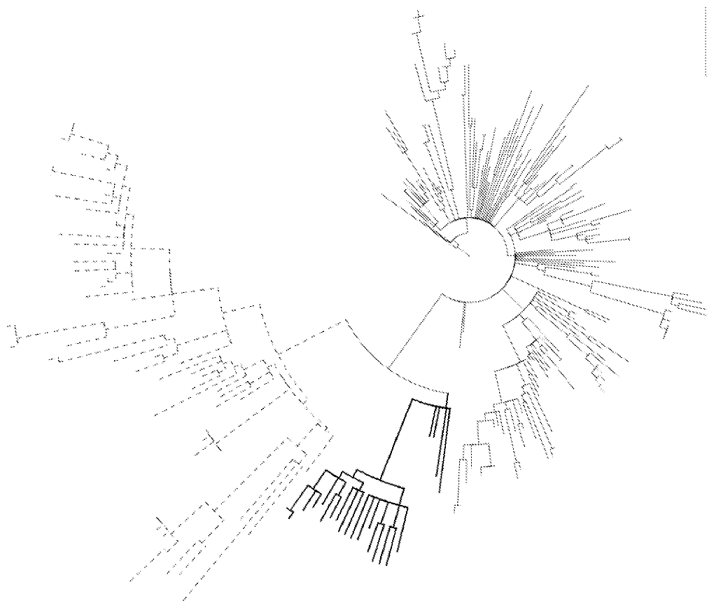

(2) The hypervirulent RT027 strains have an average resistance rate of higher than 20% to any of moxifloxacin, metronidazole or vancomycin (see Table 3). However, by defining resistant or sensitive branches based on the evolutionary relationship and the drug sensitivity test results, the RT027 strains can be further subdivided into three categories, each category having a distinctly different resistance rate. FIG. 1 shows the three drug-resistant branches of Clostridium difficile hypervirulent ribotype 027 based on the resistance to the MOX drug, wherein the strains represented by the grey dotted lines belong to Category 1 and comprise a total of 56 strains; the strains represented by the black solid lines belong to Category 2 (sensitive branch) and comprise a total of 23 strains; and the strains represented by the grey solid lines belong to Category 3 and comprise a total of 190 strains.

(3) The SNP markers in each branch are identified by:

a) screening SNP sites genome-wide;

b) skipping the site at which the bases occurring at the highest frequency for the strains of the three categories are the same; and

c) skipping the site at which there is unknown information (e.g., due to sequencing errors or insufficient sequencing coverage, the base for a strain at the site is unknown).

(4) The base type and base frequency information of the sites remaining after the screening in step (3) is analyzed, and the SNP sites at which the main bases in the same strain category have a frequency of greater than a certain threshold value (e.g., 90% or 95% or 100% etc.) are selected as the markers for distinguishing different categories of the strains, as shown in Table 1.

(5) Strains of different categories are distinguished and identified by using the SNP markers obtained in step (4) individually or in combination, depending on the application scenario.

(6) Based on the result of identification of the strain category, specific phenotypic information of the category is obtained.

(7) Clustering analysis is conducted on the 383 strains in clade2 by using the marker sites shown in Table 1, and statistics of proportions of drug-resistance is conducted on each sub-category, revealing high consistency with the categorization of RT027, as shown in Table 4.

TABLE 1
20 strain-specific SNP markers

| SNP site | Base (percentage of the bases in the category) | | | |
|---|---|---|---|---|
| (genomic | Category 1 | Category 2 | Category 3 | Category |
| position) | (56 strains) | (23 strains) | (190 strains) | specificity |
| 1029237 | A (100.0%) | G (100.0%) | G (100.0%) | Category 1 |
| 118669 | A (100.0%) | A (100.0%) | G (100.0%) | Category 3 |
| 1205250 | G (100.0%) | G (100.0%) | C (100.0%) | Category 3 |
| 1205938 | T (100.0%) | C (100.0%) | C (100.0%) | Category 1 |
| 1235096 | A (100.0%) | A (100.0%) | C (100.0%) | Category 3 |
| 1462869 | C (100.0%) | C (100.0%) | A (100.0%) | Category 3 |
| 1549858 | A (100.0%) | A (100.0%) | T (100.0%) | Category 3 |
| 1550363 | C (100.0%) | A (100.0%) | C (100.0%) | Category 2 |
| 2367860 | G (100.0%) | G (100.0%) | A (100.0%) | Category 3 |
| 2487991 | A (100.0%) | G (100.0%) | G (100.0%) | Category 1 |
| 2851331 | G (100.0%) | G (100.0%) | A (100.0%) | Category 3 |
| 2861888 | A (100.0%) | G (100.0%) | G (100.0%) | Category 1 |
| 3031309 | A (100.0%) | A (100.0%) | C (100.0%) | Category 3 |
| 3419928 | A (100.0%) | A (100.0%) | G (100.0%) | Category 3 |
| 6310 | T (100.0%) | C (100.0%) | T (100.0%) | Category 2 |
| 882348 | T (100.0%) | C (100.0%) | C (97.9%), T (2.1%) | Category 1 |
| 1602810 | C (100.0%) | C (100.0%) | G (98.9%), T (1.1%) | Category 3 |
| 1798870 | G (100.0%) | A (100.0%) | A (97.4%), G (2.6%) | Category 1 |
| 2585036 | G (100.0%) | G (100.0%) | T (97.9%), G (2.1%) | Category 3 |
| 3083454 | A (100.0%) | G (100.0%) | G (98.9%), A (1.1%) | Category 1 |

In Table 1, the location of the SNP site on the genome is the location on the whole genome sequence of Clostridium difficile CD196 strain as a reference genome.

Correspondingly, an example of the present disclosure provides a method for identifying the category of a Clostridium difficile strain, comprising obtaining base information at the site of at least one SNP marker of the SNP markers shown in Table 1 in a Clostridium difficile strain to be identified; and determining the category of the Clostridium difficile strain according to the base information.

Strains of different categories are distinguished and identified by using the SNP markers shown in Table 1 individually or in combination, depending on the application scenario.

In one case, base information is obtained at the site of at least one SNP marker of the SNP markers specific to Category 1, Category 2 or Category 3 shown in Table 1 in a Clostridium difficile strain to be identified; and it is determined whether the Clostridium difficile strain belongs to Category 1, Category 2 or Category 3 according to the base information. For example, in one example, if it is only necessary to identify whether a strain belonging to clade 2 (RT027) is a strain of Category 1, the base type of the strain at site 1029237 (or site 1205938, or site 2487991, or site 2861888) can be identified to determine whether the strain belongs to Category 1. And so on and so forth.

In another case, base information is obtained at the site of at least one SNP marker in each category of at least two categories of the SNP markers specific to Category 1, Category 2 or Category 3 shown in Table 1 in a Clostridium difficile strain to be identified; and it is determined whether the Clostridium difficile strain belongs to Category 1, Category 2 or Category 3 according to the base information. For example, in one example, if it is only necessary to identify the exact category of a strain belonging to clade 2 (RT027), the base type of the strain at two category-specific sites, such as Category 1-specific site 1205938 and Category 2-specific site 6310 (or Category 1-specific site 2487991 and Category 3-specific site 3419928, or any other combinations) can be identified, and the strain can be determined to belong to Category 3 if the result of identification of site 1205938 shows that the strain does not belong to Category 1 and the result of identification of site 6310 shows that the strain does not belong to Category 2. And so on and so forth.

In one example, the base information of the site of the SNP marker is obtained by amplifying a genomic region in which the site of the SNP marker is located by using primers adapted to specifically amplify the region, followed by sequencing the region. In the present disclosure, the primers are designed according to conventional practice in the art, and are not particularly limited. Such primers comprise a forward primer and a reverse primer, the forward primer and the reverse primer respectively specifically binding to a genomic sense strand and a genomic antisense strand flanking the SNP marker.

Table 2 below shows the forward and reverse primers for detecting some of the SNP marker sites, as well as the category specificity of the primers.

TABLE 2

| | | | Category |
|---|---|---|---|
| SNP site | Forward primer | Reverse primer | specificity |
| 1029237 | TGGATTATGCACTTGAAATATGTGAG | TGGTGTTGCCATTTCCA | Category 2, 3 |
| | (SEQ ID NO: 1) | CTG (SEQ ID NO: 3) | |
| | TGGATTATGCACTTGAAATATGTGAA | | Category 1 |
| | (SEQ ID NO: 2) | | |
| 1205938 | ATATCAAGATATGTAGCTGCTATAGC | TTTCGACTTTCTTCTGGT | Category 2, 3 |
| | (SEQ ID NO: 4) | CA (SEQ ID NO: 6) | |
| | ATATCAAGATATGTAGCTGCTATAGT | | Category 1 |
| | (SEQ ID NO: 5) | | |
| 2861888 | AGGTAATCATTTACGAATCG | TCTGACATAAGAAAATC | Category 2, 3 |
| | (SEQ ID NO: 7) | CTCCTTGT (SEQ ID NO: 9) | |
| | AGGTAATCATTTACGAATCA | | Category 1 |
| | (SEQ ID NO: 8) | | |
| 1205250 | AGCCAGGTTCTTCATTTAAGATGG | GGTTTCCTCTACCATGG | Category 1, 2 |
| | (SEQ ID NO: 10) | TTCCA (SEQ ID NO: 12) | |
| | AGCCAGGTTCTTCATTTAAGATGC | | Category 3 |
| | (SEQ ID NO: 11) | | |
| 1235096 | GTCAATTTTACAATTGTTGACAA | AGGATCAAATTCCTCAA | Category 1, 2 |
| | (SEQ ID NO: 13) | ACTCA (SEQ ID NO: 15) | |
| | GTCAATTTTACAATTGTTGACAC | | Category 3 |
| | (SEQ ID NO: 14) | | |
| 1462869 | TCTTCTCTTTTGGGTATATTAGTTGC | AGCAGCTGGATTTTCTG | Category 1, 2 |
| | (SEQ ID NO: 16) | GGT (SEQ ID NO: 18) | |
| | TCTTCTCTTTTGGGTATATTAGTTGA | | Category 3 |
| | (SEQ ID NO: 17) | | |
| 2367860 | TCGCATAAAGGTATTGCAAAACTTG | AGGTCAAAAACAGGAA | Category 1, 2 |
| | (SEQ ID NO: 19) | GTGGA (SEQ ID NO: 21) | |
| | TCGCATAAAGGTATTGCAAAACTTA | | Category 3 |
| | (SEQ ID NO: 20) | | |
| 2851331 | ACCTCCTAGTATATTATTGAG | AAAGGTGGAGGTTTTAA | Category 1, 2 |
| | (SEQ ID NO: 22) | GGA (SEQ ID NO: 24) | |
| | ACCTCCTAGTATATTATTGAA | | Category 3 |
| | (SEQ ID NO: 23) | | |
| 882348 | AGAGTCTCCAAGCATTTCAGT | ACTACTGCTGGTCATGG | Category 2, 3 |
| | (SEQ ID NO: 25) | TTCT (SEQ ID NO: 27) | |
| | AGAGTCTCCAAGCATTTCAGC | | Category 1 |
| | (SEQ ID NO: 26) | | |
| 1602810 | TCCATCAGTTCTTGCAAAGTAC | GCTTGGTGCAACAGATA | Category 1, 2 |
| | (SEQ ID NO: 28) | AGT (SEQ ID NO: 31) | |
| | TCCATCAGTTCTTGCAAAGTAG | | Category 3 |
| | (SEQ ID NO: 29) | | |
| | TCCATCAGTTCTTGCAAAGTAT | | Category 3 |
| | (SEQ ID NO: 30) | | |
| 2585036 | ACTTTTTATTGCTTATTACTTCATTG | AGGTGAGTGTGAATGTT | Category 1, 2 |
| | (SEQ ID NO: 32) | CATTTGA (SEQ ID NO: 34) | |
| | ACTTTTTATTGCTTATTACTTCATTT | | Category 3 |
| | (SEQ ID NO: 33) | | |

In Table 2, the SNP site refers to the position on the genome of the Clostridium difficile CD196 strain; the SNP marker site in the primer sequences is underlined; and the two pairs of primers used for identifying the same SNP marker site share the same reverse primer, and are only different in the last base at the 3′ end of the forward primers.

For the primers in Table 2, in actual applications, fluorescent labels of different colors can be respectively added to the two pairs of primers that recognize the same SNP marker site, and the category of the strain can be determined according to the difference in the fluorescence of two colors in the process of qPCR; alternatively, the sequence can be amplified using conventional PCR, and the SNP marker site can be determined by means of mass spectrometry or sequencing; and further alternatively, the primers can be reconstructed as hybridization probes for use in DNA chips.

The present disclosure finds use in clinical diagnosis and treatment. In one example, a method is provided for diagnosing the category of a Clostridium difficile strain in a subject infected with the strain, comprising: obtaining base information at the site of at least one SNP marker of the SNP markers according to the present disclosure in the Clostridium difficile strain from the subject; and determining the category of the Clostridium difficile strain according to the base information.

In one case, base information is obtained at the site of at least one SNP marker of the SNP markers specific to Category 1, Category 2 or Category 3 of the present disclosure in a Clostridium difficile strain from the subject infected with the strain; and it is determined whether the Clostridium difficile strain belongs to Category 1, Category 2 or Category 3 according to the base information. For example, in one example, if it is only necessary to identify whether a Clostridium difficile strain belonging to clade 2 (RT027) from the subject infected with the strain is a strain of Category 1, the base type of the strain at site 1029237 (or site 1205938, or site 2487991, or site 2861888) can be identified to determine whether the strain belongs to Category 1. And so on and so forth.

In another case, base information is obtained at the site of at least one SNP marker in each category of at least two categories of the SNP markers specific to Category 1, Category 2 or Category 3 of the present disclosure in a Clostridium difficile strain from the subject infected with the strain; and it is determined whether the Clostridium difficile strain belongs to Category 1, Category 2 or Category 3 according to the base information. For example, in one example, if it is only necessary to identify the exact category of a Clostridium difficile strain belonging to clade 2 (RT027) from the subject infected with the strain, the base type of the strain at two category-specific sites, such as Category 1-specific site 1205938 and Category 2-specific site 6310 (or Category 1-specific site 2487991 and Category 3-specific site 3419928, or any other combinations) can be identified, and the strain can be determined to belong to Category 3 if the result of identification of site 1205938 shows that the strain does not belong to Category 1 and the result of identification of site 6310 shows that the strain does not belong to Category 2. And so on and so forth.

In the present disclosure, based on the result of identification of the strain category, specific phenotypic information of the category can be obtained.

The clade 2 (hypervirulent RT027) strains have an average resistance rate of higher than 30% to any of moxifloxacin, metronidazole or vancomycin (see Table 3). However, by defining resistant or sensitive branches based on the evolutionary relationship and the drug sensitivity test results, the RT027 strains can be further subdivided into three categories, each category having a distinctly different resistance rate. The hypervirulent RT027 strains of Category 2 have a low resistance rate to moxifloxacin and metronidazole, which is only 4.35% and 0.0%, respectively, while the hypervirulent RT027 strains of Category 2 and Category 3 have a resistance rate of slightly higher than 20% to vancomycin, which is 21.74% and 21.05%, respectively. Similarly, the clade2 strains of Category 2 have a low resistance rate to moxifloxacin and metronidazole, which is only 2.08% and 3.03%, respectively, while the clade2 strains of Category 2 and Category 3 have a resistance rate of higher than 20% to vancomycin, which is 30.21% and 22.58%, respectively. Clinical subdivision of the strains to obtain phenotypic information of the corresponding category will help to improve the correct use of antibiotics, reduce the wastage of medical resources, and reduce the suffering of patients. Therefore, the category-specific SNP sites according to the present disclosure have a significant clinical application value.

TABLE 3
Antibiotics resistance rate of 269 RT027 strains

| | Resistance rate to | Resistance rate to | Resistance rate to | Average |
|---|---|---|---|---|
| | Category 1 | Category 2 | Category 3 | resistance |
| Antibiotics | (56 strains) | (23 strains) | (190 strains) | rate |
| Moxifloxacin | 96.43% | 4.35% | 98.42% | 89.96% |
| Metronidazole | 51.79% | 0.0% | 47.37% | 44.24% |
| Vancomycin | 94.64% | 21.74% | 21.05% | 36.43% |

TABLE 4
Antibiotics resistance rate of 383 clade2 strains

| | Resistance rate to | Resistance rate to | Resistance rate to | Average |
|---|---|---|---|---|
| | Category 1 | Category 2 | Category 3 | resistance |
| Antibiotics | (70 strains) | (96 strains) | (217 strains) | rate |
| Moxifloxacin | 94.29% | 2.08% | 98.62% | 73.63% |
| Metronidazole | 52.86% | 3.13% | 48.39% | 37.86% |
| Vancomycin | 92.86% | 30.21% | 22.58% | 37.34% |

Based on the phenotypic information from the above resistance rates, an example of the present disclosure provides a method for treating a subject infected with a Clostridium difficile strain, comprising obtaining base information at the site of at least one SNP marker of the SNP markers according to the present disclosure in the Clostridium difficile strain from the subject; determining the category of the Clostridium difficile strain according to the base information; and administering moxifloxacin and/or metronidazole to the subject when the category of Clostridium difficile strain comprises Category 2; and optionally, administering vancomycin to the subject when the category of the Clostridium difficile strain comprises Category 2 and/or Category 3.

The SNP markers and primers according to the present disclosure are all useful in the identification of the category of a Clostridium difficile strain, or in the diagnosis of the category of a Clostridium difficile strain from a subject infected with the strain. Therefore, an example of the present disclosure provides a use of the SNP markers and primers according to the present disclosure in the identification of the category of a Clostridium difficile strain, or in the diagnosis of the category of a Clostridium difficile strain from a subject infected with the strain.

The present disclosure has been described above with reference to specific examples, which are merely intended to aid the understanding of the present disclosure and are not intended to limit the present disclosure thereto. Several simple derivations, variations or substitutions can be made by a person skilled in the art to which the present disclosure pertains in light of the concept of the present disclosure.

What is claimed is:

1. A method for identifying the category of a Clostridium difficile strain, wherein the Clostridium difficile is Clostridium difficile clade2,
the method comprising:
obtaining a Clostridium difficile strain to be identified;
obtaining base information at a site of at least one SNP marker in the Clostridium difficile strain to be identified; and
determining a category of the Clostridium difficile strain according to the base information,
wherein the at least one SNP marker is selected from the group consisting of SNP markers in the following three categories, and combinations thereof:
(a) Category 1: an A base at genomic base position 1029237, a T base at genomic base position 1205938, an A base at genomic base position 2487991, an A base at genomic base position 2861888, a T base at genomic base position 882348, a G base at genomic base position 1798870, and an A base at genomic base position 3083454;
(b) Category 2: a C base at genomic base position 6310, and an A base at genomic base position 1550363;
(c) Category 3: a G base at genomic base position 118669, a C base at genomic base position 1205250, a C base at genomic base position 1235096, an A base at genomic base position 1462869, a T base at genomic base position 1549858, an A base at genomic base position 2367860, an A base at genomic base position 2851331, a C base at genomic base position 3031309, a G base at genomic base position 3419928, a G base at genomic base position 1602810, and a T base at genomic base position 2585036.

2. The method according to claim 1, wherein said obtaining the Clostridium difficile strain to be identified comprises:
obtaining a sample comprising the Clostridium difficile strain from a subject infected with a Clostridium difficile strain.

3. The method according to claim 1, wherein said obtaining base information at a site of at least one SNP marker in the Clostridium difficile strain to be identified comprises:
obtaining the base information at the site of at least one SNP marker in Category 1, Category 2 or Category 3 in the Clostridium difficile strain to be identified; and
wherein said determining the category of the Clostridium difficile strain according to the base information comprises:
determining whether the Clostridium difficile strain belongs to said Category 1, Category 2 or Category 3 according to the base information.

4. The method according to claim 1, wherein said obtaining base information at a site of at least one SNP marker in the Clostridium difficile strain to be identified comprises:
obtaining the base information at the site of at least one SNP marker in each category of at least two categories of Category 1, Category 2 or Category 3 in the Clostridium difficile strain to be identified; and
wherein said determining the category of the Clostridium difficile strain according to the base information comprises:
determining whether the Clostridium difficile strain belongs to said Category 1, Category 2 or Category 3 according to the base information.

5. The method according to claim 1, wherein the method obtains the base information at the site of said SNP marker by amplifying a genomic region in which the site of said SNP marker is located by using primers adapted to specifically amplify the region, followed by sequencing the region.

6. A method for treating a subject infected with a Clostridium difficile strain, wherein the Clostridium difficile is Clostridium difficile clade2,
the method comprising:
obtaining a sample comprising Clostridium difficile strain to be identified from the subject;
obtaining base information at a site of at least one SNP marker in the Clostridium difficile strain from the subject;
determining a category of the Clostridium difficile strain according to the base information,
wherein the at least one SNP marker is selected from the group consisting of SNP markers in the following three categories, and combinations thereof:
(a) Category 1: an A base at genomic base position 1029237, a T base at genomic base position 1205938, an A base at genomic base position 2487991, an A base at genomic base position 2861888, a T base at genomic base position 882348, a G base at genomic base position 1798870, and an A base at genomic base position 3083454;
(b) Category 2: a C base at genomic base position 6310, and an A base at genomic base position 1550363;
(c) Category 3: a G base at genomic base position 118669, a C base at genomic base position 1205250, a C base at genomic base position 1235096, an A base at genomic base position 1462869, a T base at genomic base position 1549858, an A base at genomic base position 2367860, an A base at genomic base position 2851331, a C base at genomic base position 3031309, a G base at genomic base position 3419928, a G base at genomic base position 1602810, and a T base at genomic base position 2585036; and
administering moxifloxacin and/or metronidazole to the subject when the category of Clostridium difficile strain comprises said Category 2.

7. The method according to claim 6, wherein the base information at the site of said SNP marker is obtained by amplifying a genomic region in which the site of said SNP marker is located by using primers adapted to specifically amplify the region, followed by sequencing the region.

8. A kit for identifying a category of a Clostridium difficile strain based on base information of SNP marker, the kit comprising:
primers comprising a forward primer and a reverse primer,
wherein the forward primer and the reverse primer specifically bind to a genomic sense strand and a genomic antisense strand flanking the SNP marker, respectively, and the forward primer and the reverse primer are adapted to specifically amplify a genomic region in which the site of said SNP marker is located.

9. The kit according to claim 8, further comprising PCR amplification components.

10. The kit according to claim 8, wherein the primers comprise a fluorescent label and are adapted for fluorescence quantitative PCR.

11. The kit according to claim 8, wherein the primers serve as hybridization probes to be immobilized on a chip to capture a sequence of the genomic region in which the site of said SNP marker is located.

12. The kit according to claim 8, wherein the forward primer has a sequence set forth as SEQ ID NO: 1, SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28, SEQ ID NO: 29, SEQ ID NO: 30, SEQ ID NO: 32, or SEQ ID NO: 33.

13. The kit according to claim 8, wherein the reverse primer has a sequence set forth as SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 31, or SEQ ID NO: 34.
